(19) United States
(12) Patent Application Publication
(10) Pub. No.: US 2003/0092758 A1
(43) Pub. Date: May 15, 2003

(54) Use of a RAR-gamma-specific agonist ligand for increasing the rate of apoptosis

(76) Inventors: Laszlo Fesus, Debrecen (HU); Zsuzsa Szondy, Debrecen (HU); Uwe Reichert, Pont-du-Loup (FR)

Correspondence Address: Norman H. Stepno, BURNS, DOANE, SWECKER & MATHIS, L.L.P., P.O. Box 1404, Alexandria, VA, 22313-1404, US

(21) Appl. No.: 10/183,518

(22) Filed: Jun. 28, 2002

Related U.S. Application Data
(63) Continuation-in-part of application No. 09/051,407, filed on Jul. 15, 1998.
Application No. 09/051,407 is a 371 of international application No. PCT/FR96/01568, filed on Oct. 8, 1996.

(30) Foreign Application Priority Data
Oct. 9, 1995 (FR) 95/12179

(51) Int. Cl. A61K 31/381; A61K 31/192

(52) U.S. Cl. 514/448; 514/569

(57) ABSTRACT

The present invention relates to the use of at least one RAR receptor agonist ligand, particularly 6-[3-(adamantan-1-yl)-4-(prop-2-ynyloxy)phenyl]naphthalene-2-carboxylic acid or 5-[(E)-3-oxo-3-(5,5,8,8-tetrahydronaphthalene-2-yl)propenyl]thiophene-2-carboxylic acid, to prepare a pharmaceutical composition for increasing the rate of apoptosis in at least one cell population in which apoptosis may be induced by activating RAR-γ receptors. The composition is particularly useful for treating a disease or disorder related to an insufficient rate of apoptosis in at least one cell population in which apoptosis may be induced by activating RAR-γ receptors. The invention further relates to the use of at least one RAR receptor agonist ligand, particularly 6-[3-(adamantan-1-yl)-4-(prop-2-ynyloxy)phenyl]naphthalene-2-carboxylic acid or 5-[(E)-3-oxo-3-(5,5,8,8-tetrahydronaphthalene-2-yl)propenyl]thiophene-2-carboxylic acid, as the active ingredient in a pharmaceutical or cosmetic composition and the use of such RAR agonist ligands to treat various disorders associated with apoptosis, differentiation, and proliferation. The compositions of the present invention may be used to prevent and/or control photo-induced or chronological aging of the skin.

### CROSS-REFERENCE TO RELATED APPLICATIONS

This application is a continuation-in-part of U.S. application Ser. No. 09/051,407, filed Jul. 15, 1998, incorporated by reference herein in its entirety and relied upon, which is a national stage filing under 35 U.S.C. §371 of International Application PCT/FR96/01568, filed Oct. 8, 1996, and claims priority to French Application No. 95/12179, filed Oct. 9, 1995.

### BACKGROUND OF THE INVENTION

1. Field of the Invention

The present invention relates to the use of specific retinoids in the preparation of a pharmaceutical composition which is intended to increase the rate of apoptosis. These retinoids can also be used in cosmetic compositions which are intended, in particular, to prevent and/or combat photo-induced or chronological aging of the skin.

2. Background of the Related Art

Two types of mechanism are involved in the death of cells. The first, which is the classical type, is termed necrosis. Morphologically, necrosis is characterized by swelling of the mitochondria and the cytoplasm and by nuclear distortion, followed by destruction of the cell and its autolysis, with the latter being accompanied by an inflammation phenomenon. Necrosis occurs in a passive and incidental manner. Tissue necrosis is generally due to the cells being subjected to a physical trauma, or due to a chemical poison, for example.

The other form of cell death is termed apoptosis (Kerr, J. F. R. and Wyllie, A. H., Br. J. Cancer, 265:239 (1972)); however, contrary to necrosis, apoptosis does not result in any inflammation phenomenon. Apoptosis has been reported to be able to take place under various physiological conditions. It is a highly selective form of cell suicide which is characterized by readily observable morphological and biochemical phenomena. Thus, condensation of the chromatin, which is or is not associated with an endonuclease activity, formation of apoptotic bodies and fragmentation of the deoxyribonucleic acid (DNA), by activation of endonucleases, into 180-200 base pair DNA fragments (these fragments can be observed by means of agarose gel electrophoresis) are, in particular, observed.

Apoptosis can be regarded as being a programmed cell death which is involved in tissue development, differentiation and renewal. It is also thought that the differentiation, growth and maturation of cells are closely linked to apoptosis and that the substances which are able to play a role in the differentiation, growth and maturation of cells are also linked to the phenomenon of apoptosis.

In the medical field, some pathological situations exhibit a modified, if not deregulated, apoptosis mechanism. Thus, it has been reported that deliberate modulation of apoptosis, by inducing it or suppressing it, can make it possible to treat a large number of diseases, more specifically diseases linked to cell hyperproliferation, as in the case of cancer, autoimmune diseases and allergies, or, on the other hand, diseases which are linked to cell disappearance, as in the case of the human immunodeficiency virus (HIV) immunodeficiency syndrome, neurodegenerative diseases (Alzheimer's disease) or excessive damage which is induced during myocardial infarction.

Specifically, it has been noted in oncology that a large number of antineoplastic drugs, such as dexamethasone, cyclophosphamide and cisplatin, are able to induce apoptosis.

In the cosmetic field, the signs of cutaneous aging essentially result from dysfunction of the principal biological mechanisms of the skin which, in particular, bring the mechanism of apoptosis into play. It is possible, therefore, to imagine that any product which induces the mechanism of apoptosis is a product which is suitable for preventing and/or combating the appearance of aging and the existing signs of aging such as large and small wrinkles.

In the field of retinoids, it is known that all-trans retinoic acid is a powerful modulator (i.e. an inhibitor or, on the other hand, a stimulator, depending on the nature of the cells which are treated) of the differentiation and proliferation of many normal or transformed cell types. For example, it inhibits the differentiation of epithelial cells such as the keratinocytes of the epidermis. It also inhibits the proliferation of many transformed cells such as melanoma cells. These effects on proliferation and differentiation can affect one and the same type of cell simultaneously, as is the case, for example, for HL-60 human promyelocytic cells; thus, it is known that proliferation of these cells is inhibited by all-trans retinoic acid and that, at the same time, their differentiation into granulocytes and their apoptosis are induced.

It is known, in a general manner, that all-trans retinoic acid acts on the differentiation and proliferation of cells by interacting with nucleoreceptors which are termed RARs (retinoic acid receptors) and which are present in the cell nucleus. To date, three subtypes of RAR receptors, termed RAR-α, RAR-β and RAR-γ, respectively, have been identified. After having bound the ligand (i.e. all-trans retinoic acid), these receptors interact with specific response elements (RARE) in the promoter region of genes which are regulated by retinoic acid. In order to bind to the response elements, the RARs heterodimerize with another type of receptor known as RXR receptors. The natural ligand of the RXRs is 9-cis-retinoic acid. The RXRs are regarded as being master regulatory proteins because they interact with other members of the steroid/thyroid receptor superfamily, such as the receptor for vitamin D3 (VDR), the receptor for triiodothyroxine (TR) and the PPARs (peroxisome proliferator activated receptors), to form heterodimers, as they do with the RARs. Furthermore, the RXRs are able to interact with specific response elements (RXRE) in the form of homodimers. These complex interactions, and the existence of numerous RAR and RXR receptors which are expressed differently depending on the tissue and the cell type, explain the pleiotropic effects of retinoids in virtually all cells.

Large numbers of synthetic structural analogues of all-trans retinoic acid or of 9-cis-retinoic acid, commonly termed “retinoids”, have so far been described in the literature. Some of these molecules are able to bind to, and specifically activate, the RARs or, on the other hand, the RXRs. Furthermore, some analogues are able to bind to, and activate, a particular subtype (α, β, or γ) of RAR receptor. Finally, other analogues do not exhibit any particular selective activity with regard to these different receptors. In this respect, and by way of example, 9-cis-retinoic acid activates both the RARs and the RXRs without any noteworthy selectivity for either of these receptors (nonspecific agonist ligand), whereas all-trans retinoic acid selectively activates the RARs (RAR-specific agonist ligand) without regard to subtype. In a general manner, and qualitatively, a given substance (or ligand) is said to be specific for a given receptor family (or with regard to a particular receptor of this family) when the substance exhibits an affinity for all the receptors of this family (or, respectively, for the particular receptor of this family) which is stronger than that which it otherwise exhibits for all the receptors of any other family (or, respectively, for all the other receptors, of this same family or not).

It has been reported that 9-cis-retinoic acid and all-trans retinoic acid are modulators of apoptosis (activator or inhibitor of apoptosis depending, in particular, on the cell type) and that 9-cis-retinoic acid is the more active of these two modulators, with it being possible to explain this observation by the fact that 9-cis-retinoic acid activates both the RARs and the RXRs, contrary to all-trans retinoic acid, which only activates the RARs.

In view of that which has been previously stated, it appears to be of interest to find novel modulators of apoptosis.

### SUMMARY AND DETAILED DESCRIPTION OF THE INVENTION

In this regard, the Applicant has just discovered that agonist ligands which are specific for receptors of the RAR-γ type are excellent inducers of apoptosis in a variety of cell types, more specifically in thymocytes.

The present invention relates to the use of at least one agonist ligand which is specific for receptors of the RAR-γ type in the preparation of a pharmaceutical composition which is intended to increase the rate of apoptosis in at least one cell population in which apoptosis can be induced by activating receptors of the RAR-γ type.

The invention also relates to the use, in a cosmetic composition, of at least one agonist ligand which is specific for receptors of the RAR-γ type as an inducer of apoptosis in at least one cell population of the skin in which apoptosis can be induced by activating receptors of the RAR-γ type.

Thus, this composition can make it possible to prevent and/or combat photo-induced or chronological aging of the skin, in particular by eliminating, by means of apoptosis, cells of the skin which exhibit a deficiency in their repair function and which accumulate over time.

The invention therefore relates, finally, to a cosmetic process for preventing and/or combating photo-induced or chronological aging of the skin, characterized in that an apoptosis-inducing cosmetic composition, such as previously described, is applied to the skin.

The pharmaceutical or cosmetic composition according to the invention comprises a physiologically acceptable medium.

Agonist ligand which is specific for receptors of the RAR-γ type is understood, according to the invention, as meaning any agonist ligand which exhibits a ratio R of the dissociation constant of this ligand for receptors of the RAR-α type over the dissociation constant of this ligand for receptors of the RAR-γ type which is greater than or equal to 10, and which induces differentiation of F9 cells.

Thus, it is known that all-trans retinoic acid and some of its analogues are able to induce the differentiation of mouse embryonic teratocarcinoma cells (F9 cells); they are therefore regarded as being agonists for the RAR receptors. The secretion of the plasminogen activator which accompanies this differentiation is an index of the biological response of the F9 cells to the retinoids (Skin Pharmacol. 3:256-267 (1990)).

The dissociation constants are determined by means of tests which are standard for the skilled person. These tests are described, in particular, in the following references: (1) “Selective Synthetic Ligands for Nuclear Retinoic Acid Receptor Subtypes” in RETINOIDS, Progress in Research and Clinical Applications, Chapter 19 (pp 261-267), Marcel Dekker Inc., edited by Maria A. Livrea and Lester Packer; (2) “Synthetic Retinoids: Receptor Selectivity and Biological Activity” in Pharmacol. Skin, Basel, Karger, 1993, Volume 5, pp 117-127; (3) “Selective Synthetic Ligands for Human Nuclear Retinoic Acid Receptors” in Skin Pharmacology, 5:57-65 (1992); (4) “Identification of Synthetic Retinoids with Selectivity for Human Nuclear Retinoic Acid Receptory” in Biochemical and Biophysical Research Communications, 186(2):977-983 (July 1992); (5) “Selective High Affinity RAR-α or RAR-β Retinoic Acid Receptor Ligands” in Mol. Pharmacol., 40:556-562.

Other characteristics, aspects, aims and advantages of the invention will become even clearer from reading the description which follows as well as the various specific examples, which are intended as an illustration and in no way as being limiting.

Agonist ligands which are specific for receptors of the RAR-γ type, and which may be mentioned, are 6-3-(1-adamantyl)-4-hydroxyphenyl)-2-naphthanoic acid, (E)-4-(1-hydroxy-1-(5,6,7,8-tetrahydro-5,5,8,8-tetramethyl-2-naphthyl)-2-propenyl)benzoic acid, 4-[(E)-2-(3-(1-adamantyl)-4-hydroxyphenyl)-1-propenyl]-benzoic acid, 5′,5′,8′,8′-tetramethyl-5′,6′,7′,8′-tetrahydro-[2,2′]binaphthalenyl-6-carboxylic acid, 2-(5,5,8,8-tetramethyl-5,6,7,8-tetrahydro-naphthalen-2-yl)-benzo[b]thiophene-6-carboxylic acid, 4-(5,5,8,8-tetramethyl-5,6,7,8-tetrahydro-naphtho[2,3-b]thiophen-2-yl)benzoic acid, 6-(5,5,8,8-tetramethyl-5,6,7,8-tetrahydro-naphthalene-2-carbonyl)naphthalene-2-carboxylic acid, 3,7-dimethyl-7-(1,2,3,4-tetrahydro-1,4a,9b-trimethyl-1,4-methano-dibenzofuran-8-yl)-2,4,6-heptatrienoic acid, 6-(1,2,3,4-tetrahydro-1,4a,9b-trimethyl-1,4-methano-dibenzofuran -8-yl)-naphthalene-2-carboxylic acid, 6-[hydroxyimino-(5,5,8,8-tetramethyl-5,6,7,8-tetrahydro-naphthalen-2-yl)-methyl]naphthalene-2-carboxylic acid, 4-[(6-hydroxy-7-(1-adamantyl)-2-naphthyl]benzoic acid, 5-(5,5,8,8-tetramethyl-5,6,7,8-tetrahydro-anthracen-2-yl)-thiophene-2-carboxylic acid, (−)-6[hydroxy-(5,5,8,8-tetramethyl-5,6,7,8-tetrahydro-naphthalen-2-yl)-methyl]-naphthalene-2-carboxylic acid, 6-(3-adamantan-1-yl-4-prop-2-ynyloxy-phenyl)-naphthalene -2-carboxylic acid, 4-[(2-oxo-2-(5,5,8,8-tetramethyl-5,6,7,8-tetrahydro-naphthalen-2-yl)-ethoxy]-benzoic acid, 4-[2-oxo-2-(5,5,8,8-tetramethyl -5,6,7,8-tetrahydro-naphthalen-2-yl)-acetylamino]-benzoic acid, 4-[2-fluoro-2-(5,5,8,8-tetramethyl-5,6,7,8-tetrahydro-naphthalen-2-yl)-acetylamino]-benzoic acid, 6-[3-(1-adamantyl-4-(2-hydroxypropyl)phenyl]-2-naphthoic acid, 5-[3-oxo-3-(5,5,8,8-tetramethyl-5,6,7,8-tetrahydro-naphthalen-2-yl)-propenyl]-thiophene-2-carboxylic acid, 6-[3-(1-adamantyl-4-(2,3-di-hydroxypropyl)phenyl]-2-naphthoic acid, 4-[3-hydroxy-(5,6,7,8-tetrahydro-5,5,8,8-tetramethyl-2-naphthyl)-1-propynyl]-benzoic acid, 4-[3-oxo-3-(5,5,8,8-tetramethyl-5,6,7,8-tetrahydro-naphthalen -2-yl)-prop-1-ynyl]benzoic acid, 4-[(3-(1-methylcyclohexyl)-4-hydroxyphenyl)ethenyl]-benzoic acid, 4-[(E)2-(3-(1-adamantyl)-4-hydroxyphenyl)-ethenyl]-benzoic acid, 4-[3-(1-adamantyl)-4-hydroxyphenylethynyl)-benzoic acid, 5-[3-(1-adamantyl)-4-hydroxyphenyethynyl]-2-thiophenecarboxylic acid, 5-[3-(1-adamantyl)-4-methoxyphenylethynyl]-2-thiophene-carboxylic acid, 4-[2-(3-tert-butyl -4-methoxyphenyl)-propenyl]benzoic acid, 4-{2-[4-methoxy-3-(1-methylcyclohexyl)phenyl]-propenyl-benzoic acid, 6-[3-(1-adamantyl)-4-(3-methoxy-2-hydroxypropyl)phenyl]-2-naphthoic acid, 2-hydroxy-4-[3-hydroxy-3-(5,6,7,8-tetrahydro-5,5,8,8-tetramethyl-2-naphthyl)-1-propynyl]-benzoic acid, 6-(5,5,8,8-tetramethyl-5,6,7,8-tetrahydro-naphthalen-2-yloxy)-naphthalene-2-carboxylic acid, 6-(5,5,8,8-tetramethyl-5,6,7,8-tetrahydro-naphthalen-2-ylsulphanyl)-naphthalene -2-carboxylic acid, 4-[2-propoxyimino-2-(5,5,8,8-tetramethyl-5,6,7,8-tetrahydro-naphthalen -2-yl)-acetylamino]benzoic acid, 6-(5,5,8, 8-tetramethyl-5,6,7,8-tetrahydro-naphthalen-2-ylamino)naphthalene-2-carboxylic acid, 1-methyl-4-(5,5,8,8-tetramethyl-5,6,7,8-tetrahydroanthracen-2-yl)-1H-pyrrole-2-carboxylic acid, 2-methoxy-4-(5,5,8,8-tetramethyl-5,6,7,8-tetrahydro-anthracen-2-yl)-benzoic acid, 4-[2-nonyloxyimino-2-(5,5,8,8-tetramethyl-5,6,7,8-tetrahydro-naphthalen-2-yl)-acetylamino]-benzoic acid, (−)-2-hydroxy-4-[3-hydroxy-3-(5,5,8,8-tetramethyl -5,6,7,8-tetrahydro-naphthalen-2-yl)-prop-1-ynyl]-benzoic acid, (+)-2-hydroxy-4 [3-hydroxy-3-(5,5,8,8-tetramethyl-5,6,7,8-tetrahydro-naphthalen-2-yl)-prop-1-ynyl]-benzoic acid, 2-hydroxy-4-[3-hydroxy-3-(5,5,8,8-tetramethyl-5,6,7,8-tetrahydro-naphthalen-2-yl)-but-1-ynyl]-benzoic acid, 6-(3-bromo-5,5,8,8-tetramethyl-5,6,7,8-tetrahydro-naphthalen-2-yloxy)-naphthalene-2-carboxylic acid, 3-[(5,6,7,8-tetrahydro-5,5,8,8-tetramethyl-2-naphthyl)-2H-1-benzopyran]-7-carboxylic acid, 4-[3-(3,5-di-tert-butyl-4-hydroxy-phenyl)-prop-1-ynyl]-benzoic acid, 4-[3-(5,5,8,8-tetramethyl-5,6,7,8-tetrahydro-naphthalen-2-yl)-prop-1-ynyl]-benzoic acid, 4-[3-(5,6,7,8-tetrahydro-5,5,8,8-tetramethyl-2-naphthyl)-1-propynyl]-salicylic acid, 4-[{3-(1-adamantyl)-4-(2-hydroxyethyl)phenyl}ethynyl] benzoic acid, and 4-[{3-(1-adamantyl)-4-(3-hydroxypropyl)phenyl}ethynyl]-benzoic acid.

Preference is given, in the present invention, to using agonist ligands which are specific for receptors of the RAR-γ type which exhibit a ratio R which is greater than or equal to 50. As such, preference is given to using 6-3-(1-adamantyl)-4-hydroxyphenyl)-2-naphthanoic acid, 6-(5,5,8,8-tetramethyl-5,6,7,8-tetrahydro-naphthalene-2-carbonyl)-naphthalene-2-carboxylic acid, 6-(1,2,3,4-tetrahydro-1,4a,9b-trimethyl-1,4-methano-dibenzofuran-8-yl)-naphthalene-2-carboxylic acid, 6-[hydroxyimino-(5,5,8,8-tetramethyl-5,6,7,8-tetrahydro-naphthalen -2-yl)-methyl]-naphthalene-2-carboxylic acid, 5-(5,5,8,8-tetramethyl -5,6,7,8-tetrahydro-anthracen-2-yl)-thiophene-2-carboxylic acid, (−)-6-[hydroxy -(5,5,8,8-tetramethyl-5,6,7,8-tetrahydro-naphthalen-2-yl)-methyl]-naphthalene-2-carboxylic acid, 6-[3-(1-adamantyl-4-(2-hydroxypropyl)phenyl]-2-naphthoic acid, 6-[3-(1-adamantyl-4-(2,3-di-hydroxypropyl)phenyl]-2-naphthoic acid, 4-[3-(1-adamantyl)-4-hydroxyphenylethynyl]-benzoic acid, 5-[3-(1-adamantyl)-4-hydroxyphenylethynyl]-2-thiophenecarboxylic acid, 5-[3-(1-adamantyl)-4-methoxyphenylethynyl]-2-thiophenecarboxylic acid, 6-[3-(1-adamantyl)-4-(3-methoxy-2-hydroxypropyl)phenyl]-2-naphthoic acid, 1-methyl-4-(5,5,8,8-tetramethyl -5,6,7,8-tetrahydro-anthracen-2-yl)-1H-pyrrole-2-carboxylic acid, (−)-2-hydroxy-4-[3-hydroxy-3-(5,5,8,8-tetramethyl-5,6,7,8-tetrahydro-naphthalen-2-yl)-prop -1-ynyl]-benzoic acid and 2-hydroxy-4-[3-hydroxy-3-(5,5,8,8-tetramethyl -5,6,7,8-tetrahydronaphthalen-2-yl)-but-1-ynyl)-benzoic acid.

6-3-(1-Adamantyl)-4-hydroxyphenyl)-2-naphthanoic acid is particularly preferred. Also particularly preferred are 6-[3-(adamantan-1-yl)-4-(prop-2-ynyloxy)phenyl]naphthalene-2-carboxylic acid and 5-[(E)-3-oxo-3-(5,5,8,8-tetrahydronaphth-2-yl)propenyl]thiophene-2-carboxylic acid.

Thus, it will be possible to use the pharmaceutical composition comprising the agonist ligand which is specific for receptors of the RAR-γ type when it is necessary to increase the rate of apoptosis. Naturally, this effect will only be achieved in cell populations in which apoptosis can be induced by activating receptors of the RAR-γ type and therefore, in particular, in which receptors of the RAR-γ type are present, as is the case, more specifically, in cells derived from the thymus.

It may prove to be necessary to increase the rate of apoptosis in two cases in the main. The first case relates to diseases or disorders which are linked to an inadequate rate of apoptosis. The second case relates to treatments which are required during a transplantation in order to decrease the effects tending to reject the transplanted organ. Thus, it is possible to consider increasing immunotolerance to the transplanted organ by decreasing the immune response of the T cells by means of inducing their rate of apoptosis.

Diseases or disorders which are linked to an inadequate rate of apoptosis, and which may more specifically be mentioned, are disorders which are due to precancerous conditions or cancers which can be the consequence of a proliferation of certain cell populations, autoimmune diseases, allergies or inflammatory reactions in which the number of cells causing damage is observed to be too high, or else in certain viral infections in which proteins of the virus have an antiapoptotic effect. Thus, in the case of autoimmune diseases, more specific mention may be made of insulin-dependent diabetes, active chronic hepatitis, rheumatoid arthritis, pemphigus, multiple sclerosis, myasthenia, systemic lupus erythematosus, Crohn's disease and psoriasis. Actinic keratosis may be mentioned with regard to precancerous conditions. In the case of cancer, more specific mention may be made of lymphomas, carcinomas, such as cancer in the ENT sphere, and hormone-dependent tumours, such as ovarian cancers. Of the abovementioned viral infections, those which may in particular be cited are herpes viruses, adeno viruses and variola viruses (poxviruses). Allergies or inflammatory reactions which may be mentioned are contact eczema, atopic eczema, asthma and urticarias.

In addition to their application in diseases associated with inadequate apoptosis due to the ability to stimulate RARγ receptors, two of the compounds of the present invention, 6-[3-(adamantan-1-yl)-4-(prop-2-ynyloxy)phenyl] naphthalene-2-carboxylic acid and 5-[(E)-3-oxo-3-(5,5,8,8-tetrahydronaphthalene-2-yl)propenyl]thiophene-2-carboxylic acid, also stimulate RARα and RARβ receptors and are further suited for additional fields of treatment, These two compounds may be used for treating dermatological complaints associated with a keratinization disorder characterized by abnormalities of differentiation and proliferation, in particular simple acne, comedones, polymorphonuclear leukocytes, rosacea, nodulocystic acne, acne conglobata, senile acne and secondary acne such as solar, medication-related or professional acne; and for treating other types of keratinization disorders, in particular ichthyosis, ichthyosiform states, Darier's disease, palmoplantar keratoderma, leukoplasias and leukoplasiform states, and cutaneous or mucous (buccal) lichen. Other disorders to which these two compounds of the present invention are applicable are dermatological complaints with an inflammatory immunoallergic component, with or without cell proliferation disorder, and, in particular, all forms of psoriasis, whether this is cutaneous, mucous or ungual psoriasis, and even psoriatic rheumatism, or alternatively cutaneous atopy, such as eczema or respiratory atopy or alternatively gingival hypertrophy. All dermal or epidermal proliferations, whether benign or malignant and whether they are of viral origin or otherwise, such as common warts, flat warts and verruciform epidermodysplasia, oral or florid papillomatoses, T lymphoma and proliferations which may be induced by ultraviolet radiation, in particular in the case of basocellular and spinocellular epithelioma, as well as any pre-cancerous skin lesion such as keratoacanthomas may be mentioned as disorders to which these two compounds of the invention are applicable. These two compounds of the present invention can be used for treating other dermatological disorders such as immune dermatitis such as lupus erythematosus, immune bullosis and collagen diseases such as scleroderma, for treating dermatological or general complaints with an immunological component, and for treating certain ophthalmological disorders, in particular corneopathies. Other disorders which may be treated by these two compounds of the present invention are dermal or epidermal atrophy produced by local or general administration of corticisteroid or any other cutaneous atrophy. These two compounds of the present invention are suited for the treatment of any complaint of viral origin on the skin or generally and for the treatment of skin disorders due to exposure to UV radiation, as well as for repairing or combating ageing of the skin, including both light-induced or chronological ageing, or for reducing actinic keratoses and pigmentations, or any pathologies associated with chronological or actinic ageing, such as xerose. These two compounds of the present invention may also be used to combat disorders of sebaceous function such as the hyperseborrhoea of acne or simple seborrhoea, and to prevent or treat cicatrization disorders or to prevent or repair stretchmarks. Other disorders which can be treated by these two compounds of the present invention are pigmentary disorders such as hyperpigmentation, hypopigmentation, or vitiligo; disorders of lipid metabolism, such as obesity, hyperlipidemia, non-insulin-dependent diabetes; and inflammatory complaints such as arthritis. These two compounds of the present invention are further suitable for the treatment or prevention of cancerous or pre-cancerous states of cancers; the prevention or treatment of alopecia of various origins, in particular alopecia due to chemotherapy or radiation; and the treatment of immune complaints, such as autoimmune diseases, for instance type 1 diabetes mellitus, multiple sclerosis, lupus and lupus-type complaints, and asthma. Other disorders which can be treated by these two compounds of the invention are complaints of the cardiovascular system such as arteriosclerosis or hypertension, as well as non-insulin-dependent diabetes.

The composition according to the invention can be administered by the enteral, parenteral, topical or ocular route and comprises the selected agonist ligand, in an amount effective to increase the rate of apoptosis in at least one cell type population in which apoptosis is induced by activating receptors of the RAR-γ type, and a pharmaceutically or cosmetically acceptable carrier. Alternatively, when the selected active agent is one of the two specific compounds named in the preceding paragraph, it may be present in the composition in an amount effective to stimulate RARα and RARβ receptors, that is, an amount effective to treat or prevent one of the conditions specified in the preceding paragraph. Preference is given to packaging the pharmaceutical composition in a form which is suitable for administration by the systemic route (for injection or perfusion).

When administered by the enteral route, the composition, more specifically the pharmaceutical composition, can be in the form of tablets, hard gelatin capsules, coated tablets, syrups, suspensions, solutions, powders, granules, emulsions, microspheres or nanospheres, or lipid or polymeric vesicles which permit a controlled release. When administered by the parenteral route, the composition may be in the form of solutions or suspensions for perfusion or injection.

The agonist ligands which are specific for receptors of the RAR-γ type as well as the compounds 6-3[adamantan-1-yl)-4-(prop-2-ynyloxy)phenyl]naphthalene-2-carboxylic acid and 5-[(E)-3-oxo-3-(5,5,8,8-tetrahydronaphthalene-2-yl)propenyl]thiophene-2-carboxylic acid which also stimulate RARα and RARβ receptors, and which are used in accordance with the invention are generally administered in a daily dose of from 0.01 mg/kg to 100 mg/kg of bodyweight, with the dose being given in from 1 to 3 administrations.

When administered by the topical route, the pharmaceutical or cosmetic composition according to the invention is more specifically intended for treating the skin and the mucous membranes and can be in the form of ointments, creams, milks, pomades, powders, imbibed buffers, solutions, gels, sprays, lotions or suspensions. It may also be in the form of microspheres or nanospheres, or lipid or polymeric vesicles, or of polymeric patches and hydrogels, which permit a controlled release. This composition, which is administered by the topical route, can be present either in anhydrous form or in aqueous form.

When administered by the ocular route, the composition is chiefly in the form of eye drops.

The agonist ligands which are specific for receptors of the RAR-γ type, as well as the compounds 6-3[adamantan-1-yl)-4-(prop-2-ynyloxy)phenyl]naphthalene-2-carboxylic acid and 5-[(E)-3-oxo-3-(5,5,8,8-tetrahydronaphthalene-2-yl)propenyl]thiophene-2-carboxylic acid, are used by the topical or ocular route at a concentration which is generally between 0.001% and 10% by weight, preferably between 0.1 and 1% by weight, based on the total weight of the composition.

The present invention finally relates to a process for preventing and/or combating photo-induced or chronological aging of the skin, characterized in that a cosmetic composition which induces apoptosis such as previously described, that is to say which comprises at least one agonist ligand which is specific for receptors of the RAR-γ type or at least one of the compounds 6-3[adamantan-1-yl)-4-(prop-2-ynyloxy)phenyl]naphthalene-2-carboxylic acid and 5-[(E)-3-oxo-3-(5,5,8,8-tetrahydronaphthalene-2-yl)propenyl]thiophene-2-carboxylic acid, in a cosmetically acceptable medium, is applied to the skin.

Naturally, the compositions as previously described may additionally comprise inert or even pharmacodynamically active additives or combinations of these additives, in particular: wetting agents; depigmenting agents such as hydroquinone, azelaic acid, caffeic acid or kojic acid; emollients; moisturizing agents such as glycerol, PEG 400, thiamorpholinone and its derivatives, or urea; antiseborrhoeic agents or anti-acne agents, such as S-carboxymethylcysteine, S-benzyl-cysteamine, and their salts or derivatives, or benzoyl peroxide; antifungal agents such as ketoconazole or the polymethylene-4,5-isothioazolidonin-3-ones; antibacterial agents, carotenoids and, in particular, β-carotene; antipsoriatic agents such as anthralin and its derivatives; and, finally, eicosa-5,8,11,14-tetraynoic and eicosa-5,8,11-trynoic acids, and their esters and amides.

These compositions may also comprise taste-improving agents, preservatives such as esters of parahydroxybenzoic acid, stabilizers, moisture-regulating agents, pH-regulating agents, agents for modifying the osmotic pressure, emulsifying agents, UV-A and UV-B filters, antioxidants, such as α-tocopherol, butylhydroxyanisole or butylhydroxytoluene.

Naturally, the skilled person will ensure that the possible compounds to be added to these compositions is/are selected such that the advantageous properties which are intrinsically attached to the present invention are not altered, or are not substantially altered, by the addition which is envisaged.

The following examples are intended to illustrate the present invention, but are in no way limiting.

### EXAMPLE 1

This experiment demonstrates the in vivo efficacy of an agonist ligand which is specific for RAR-γ as an inducer of apoptosis.

4-week-old male NMRI mice (sold by LATI, Gödöllo, Hungary) were used. In order to induce apoptosis in the thymus, these male mice were treated by a single injection with either 0.5 mg of dexamethasone, or 0.5 mg of 6-3-(1-adamantyl)-4-hydroxyphenyl)-2-naphthanoic acid, dissolved in a mixture of 0.8 ml of physiological saline and 0.2 ml of ethanol.

Dexamethasone is a well-known inducer of apoptosis. Thus, involution of the thymus is observed: i.e. a decrease of approximately 75% in the weight of the thymus at 48 hours after the abovementioned treatment. A similar effect is observed with 6-3-(1-adamantyl)-4-hydroxyphenyl)-2-naphthanoic acid, with, in this case a decrease of approximately 60% in the weight of the thymus being observed at 48 hours after treatment.

In addition, in a similar manner to this test, thymus samples were removed from untreated or treated animals at different times after the treatment. After washing and homogenization, their transglutaminase activity was determined by detecting the incorporation of [³H]putrescine into N,N′-dimethylcasein. The activity of the transglutaminase is calculated in nmoles of [³H]putrescine incorporated into the protein per hour.

Tissue transglutaminase has been reported to be one of the elements involved in bringing about apoptosis [Piacentini, M. et al. (1994), “Apoptosis: The Molecular Basis of Apoptosis in Disease,” Curr. Comm. in Cell & Mol. Biol. 8 (Tomei L. D. and Cope, F. O. ed), pp. 143-165].

Thus, it is observed that the above-described involution of the thymus results from treating the mouse with a RAR-γ -specific agonist ligand and is an event which is concomitant with, and proportional to, the activity of the tissue transglutaminase.

Furthermore, this involution of the thymus is correlated with the appearance of DNA fragments following the analysis, by agarose gel electrophoresis, of the DNA which is recovered from this treated thymus (details of the procedure are given in Example 2).

### EXAMPLE 2

This example demonstrates the in vitro efficacy of a RAR-γ -specific agonist ligand as an inducer of apoptosis as compared with other types of retinoid.

Culture and Preparation of the Cells

Thymocyte suspensions are prepared from the thymus glands of untreated four-week-old male NMRI mice (sold by LATI, Gödöllo, Hungary). The medium employed is Sigma RPMI 1640 medium which is supplemented with Gibco foetal calf serum, 2 mM glutamine and 100 IU of penicillin and 100 μg of streptomycin/ml. The thymocytes are then washed, and diluted in order to obtain a final concentration of 10⁷ cells/ml, before being incubated at 37° C. in a humidified incubator under an atmosphere of 5% CO₂ and 95% air. Death of the cells is measured by the uptake of trypan blue.

Qualitative and Quantitative Analysis of the DNA

The thymocytes are incubated in 24 wells with various compounds to be tested at different concentrations. After 6 hours of incubation, 0.8 ml of the cell suspensions was lysed by adding 0.7 ml of lysis buffer containing 0.5% (v/v) Triton X-100, 10 mM Tris, 20 mM EDTA, pH 8.0, before being centrifuged at 13,000 g for 15 minutes.

Quantitative Analysis of the DNA: The DNA contained in the supernatant (the fragments) and the pellet (intact chromatin) was precipitated with an equivalent quantity of 10% trichloroacetic acid, resuspended in 5% trichloroacetic acid and then quantified using the diphenylamine reagent (Burton, K. Biochem. J., 62:315-322 (1956)).

Qualitative Analysis of the DNA: In parallel, the supernatant was precipitated overnight in ethanol containing 0.15 mM NaCl. The pellets are redissolved in a buffer containing 10 mM Tris, 1 mM EDTA, pH 8.0, and these solutions are then treated with RNase; they are then sequentially extracted with an equal volume of phenol and chloroform/isoamyl alcohol (24/1), after which the DNA is precipitated in ethanol before being electrophoresed for 3 hours at 60 V in a 1.8% agarose gel. The DNA fragments were then visualized with UV light after the gel had been stained with ethidium bromide. The gels which are obtained present the picture of a ladder of DNA fragments which are multiples of from 180 to 200 base pairs and which are typical of an apoptosis induction. Throughout the experiments, the degree of fragmentation correlates with the number of cells which are dead and positive to the trypan blue test.

The results of the quantitative analysis are assembled in Table 1 below.

TABLE 1

| | | | | Quantities of | |
|---|---|---|---|---|---|
| | | RAR-γ | | compounds | % of DNA |
| Compounds | RAR-α- Kd | Kd | R | (nM) | fragments |
| ATRA | 15.5 | 3 | 5.16 | 10⁻⁸ | 2 |
| | | | | 10⁻⁶ | 4 |
| 9-cisRA | 7 | 17 | 0.41 | 10⁻⁸ | 4 |
| | | | | 10⁻⁶ | 15 |
| CD437 | 6500 | 77 | 84.42 | 10⁻⁸ | 12 |
| | | | | 10⁻⁶ | 22 |
| CD666 | 2240 | 68 | 32.94 | 10⁻⁸ | 10 |
| | | | | 10⁻⁶ | 20 |
| CD2325 | 1144 | 53 | 21.58 | 10⁻⁸ | 5 |
| | | | | 10⁻⁶ | 20 |
| CD2019 | 1100 | 160 | 6.87 | 10⁻⁸ | 0 |
| | | | | 10⁻⁶ | 15 |

ATRA is all-trans retinoic acid

9-cisRA is 9-cis-retinoic acid

CD437 is 6-3-(1-adamantyl)-4-hydroxyphenyl)-2-naphthanoic acid,

CD666 is (E)-4-(1-hydroxy-1-(5,6,7,8-tetrahydro-5,5,8,8-tetramethyl-2-naphthyl)-2-propenyl)benzoic acid,

CD2325 is 4-[(E)-2-(3-(1-adamantyl)-4-hydroxyphenyl)-1-propenyl]benzoic acid,

CD2019 is 6-(3-(1-methylcyclohexyl)-4-methoxyphenyl)-2-naphthanoic acid

The percentage of DNA fragments in this table corresponds to the difference between the percentage of DNA fragments obtained in treated thymocytes and the percentage of DNA fragments obtained in nontreated thymocytes (basal rate of apoptosis for these thymocytes).

These results demonstrate that the percentage appearance of DNA fragments increases as R increases. Thus, the apoptosis-inducing effect increases as the specificity of the ligand for RAR-γ increases.

### EXAMPLE 3

### Synthesis of 6-[3-(adamantan-1-yl)-4-(prop-2-ynyloxy)phenyl]naphthalene-2-carboxylic acid

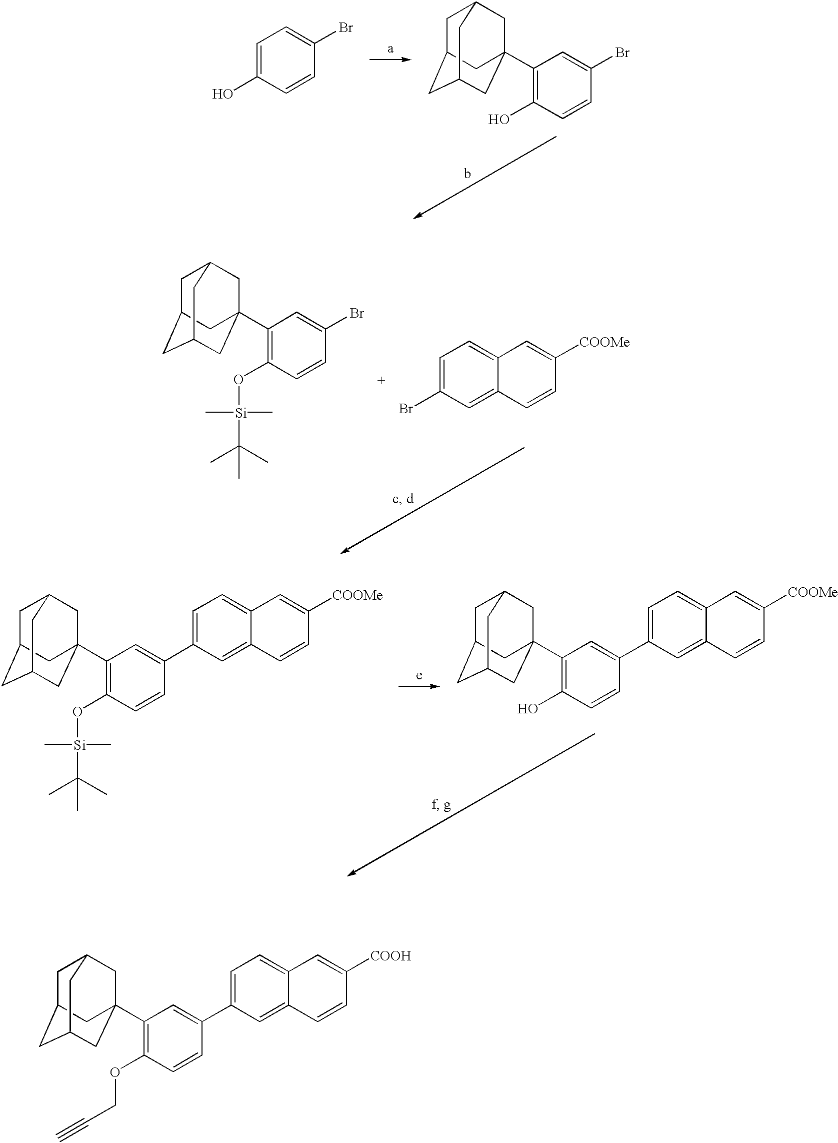

a. Ad-OH, H₂SO₄, 63%; b. TBSCl, Et₃N, DMAP, 83%; c. Mg, ZnCl₂; d. NiCl₂dppe, 54%; e. TBAF, 95%; f. NaH, propargylBr, 77%; g. NaOH, H₂O/MeOH, 89%.

Procedure

a. Preparation of 2-(adamantan-1-yl)-4-bromophenol: 51.9 g (0.3 mol) of 4-bromophenol and 45.7 g (0.3 mol) of adamantyl alcohol are dissolved in 150 ml of dichloromethane. 15 ml of concentrated sulfuric acid are added, and the mixture is stirred for 48 hours at ambient temperature and then concentrated under reduced pressure. The residue is diluted with water and then neutralized with dilute sodium hydroxide solution. The precipitate observed is filtered off, then dissolved in 200 ml of THF and dried with magnesium sulfate. The residue is purified by chromatography on a silica column (eluent: 90 heptane/10 ether) to produce the desired product (m=58 g; Y=63%).

b. Preparation of [2-(adamantan-1-yl)-4-bromophenoxy](tert-butyl)dimethylsilane: 49.5 g (161 mmol) of 2-(adamantan-1-yl)-4-bromophenol and 24.7 ml (177 mmol) of triethylamine are dissolved in 400 ml of DMF. 800 mg (6.5 mmol) of DMAP are added and then a solution of 25.2 g (167 mmol) of tert-butyldimethylsilyl chloride in 100 ml of DMF is slowly added. The mixture is stirred for 24 hours and then it is poured into 1 liter of water and extracted with 2 fractions of 300 ml of ethyl ether. The combined organic phases are rinsed three times with water and with an ammonium chloride solution. The residue is purified by chromatography on a silica column (eluent: heptane) to produce a colorless oil (m=56.1 g, Y=83%).

c, d. Preparation of methyl 6-[3-(adamantan-1-yl)-4-(tert-butyldimethylsilanyloxy)phenyl]naphthalene-2-carboxylate: 56 g (133 mmol) of [2-(adamantan-1-yl)-4-bromophenoxy](tert-butyl)dimethylsilane and 5.7 ml (66.5 mmol) of 1,2-dibromoethane are dissolved in 150 ml of anhydrous THF. This solution is slowly added to a suspension of 4.85 g (200 mmol) of magnesium in 200 ml of THF. The mixture is heated at reflux for 2 hours and then 18 g (133 mmol) of dry ZnCl₂ are added. The mixture is stirred for 2 hours at ambient temperature. 17.6 g (66.5 mmol) of methyl 6-bromo-2-naphthoate are added, the mixture is diluted with an additional 100 ml of THF, and 505 mg (0.96 mmol) of NiCl₂dppe (NiCl2-1,2-bis(diphenylphosphino)ethane) are added. The reaction mixture is stirred for 24 hours at ambient temperature and is then treated with a saturated aqueous ammonium chloride solution. After extraction with ethyl acetate, the residue is purified by chromatography on a silica column (eluent: 30 heptane/70 dichloromethane) to produce the desired product (m=19 g, Y=55%).

e. Preparation of methyl 6-[3-(adamantan-1-yl)-4-hydroxyphenyl]naphthalene-2-carboxylate: 19 g (36.1 mmol) of methyl 6-[3-(adamantan-1-yl)-4-(tert-butyldimethylsilanyloxy)phenyl]naphthalene-2-carboxylate are dissolved in 250 ml of THF. 40 ml (40 mmol) of a 1M tetrabutylammonium fluoride solution are added, and the reaction mixture is stirred for 1 hour and then treated with a saturated aqueous ammonium chloride solution. After extraction with ethyl acetate, the residue is purified by chromatography on a silica column (eluent: 80 heptane/20 dichloromethane) to produce the desired product (m=14.2 g, Y=95%).

f. Preparation of methyl 6-[3-(adamantan-1-yl)-4-(prop-2-ynyloxy)phenyl]naphthalene-2-carboxylate: 1.5 g (3.6 mmol) of methyl 6-[3-(adamantan-1-yl)-4-hydroxyphenyl]naphthalene-2-carboxylate are dissolved in 20 ml of DMF. 160 mg (4 mmol) of 60% sodium hydride are added and the mixture is stirred for 1 hour. 360 ml (4 mmol) of propargyl bromide are then added and the mixture is stirred for a further 1 hour. After the usual treatment, the residue is purified by chromatography on a silica column (eluent: 20 ethyl acetate/80 heptane) to produce a white solid (M.p.=189° C., m=1.68 g, Y=77%).

g. Preparation of 6-[3-(adamantan-1-yl)-4-(prop-2-ynyloxy)phenyl]naphthalene-2-carboxylic acid: 1.54 g (3.4 mmol) of methyl 6-[3-(adamantan-1-yl)-4-(prop-2-ynyloxy)phenyl]naphthalene-2-carboxylate are dissolved in 40 ml of methanol, and 10 ml of 1N NaOH are added. The mixture is brought to reflux for 1 hour and then cooled, concentrated and acidified to pH 1 by addition of hydrochloric acid. After extraction with ethyl ether, the crude product is triturated with heptane and then filtered off. A white solid is obtained (M.p.=249° C., m=1.32 g, Y=89%).

### EXAMPLE 4

### Synthesis of 5-[(E)-3-oxo-3-(5,5,8,8-tetramethyl-5,6,7,8-tetrahydronaphthalene-2-yl)propenyl]thiophene-2-carboxylic acid

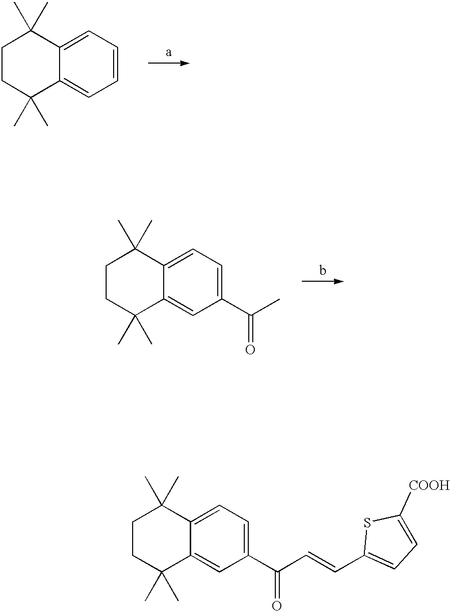

a. AlCl₃, AcCl, 78%; b. 5-CHO-thiophene-2-COOH, NaOH, 54%

Procedure

a. Preparation of 1-(5,5,8,8-tetramethyl-5,6,7,8-tetrahydronaphth-2-yl)ethanone: 29 ml (408 mmol) of acetyl chloride are dissolved in 90 ml of nitrobenzene and the mixture is cooled to 0° C. 54 g (404 mmol) of aluminum chloride are added portionwise. A solution of 75 g (400 mmol) of 5,5,8,8-tetramethyl-5,6,7,8-tetrahydronaphthalene in 130 ml of nitrobenzene is added dropwise. The reaction mixture is stirred for 3 hours at ambient temperature, and is then poured onto ice and extracted with dichloromethane. The organic phase is washed with a sodium bicarbonate solution and then dried and evaporated under reduced pressure. The residue is then distilled under reduced pressure (P=0.7 mbar), the nitrobenzene distilling at 44° C. and the product at 100° C. A thick oil is obtained (m=72 g; Y=78%).

b. Preparation of 5-[(E)-3-oxo-3-(5,5,8,8-tetramethyl-5,6,7,8-tetrahydronaphthalene-2-yl)propenyl]thiophene-2-carboxylic acid: 2.3 g (10 mmol) of 1-(5,5,8,8-tetramethyl-5,6,7,8-tetrahydronaphth-2-yl)ethanone and 1.7 g (10 mmol) of 5-formylthiophene-2-carboxylic acid are dissolved in 70 ml of methanol. 40 ml of a 1N sodium hydroxide solution are added and the reaction mixture is stirred for 48 hours at ambient temperature. The mixture is acidified by addition of hydrochloric acid and then the mixture is extracted with ethyl ether. The residue is recrystallized from ether (M.p.=204° C., m=2 g, Y=54%).

### EXAMPLE 5

Demonstration of RAR agonist activity of 6-[3-(adamantan-1-yl)-4-(prop-2-ynyloxy)phenyl]naphthalene-2-carboxylic acid and 5-[(E)-3-oxo-3-(5,5,8,8-tetrahydronaphthalene-2-yl)propenyl]thiophene-2-carboxylic acid.

The activity of RAR receptors by the compounds synthesized in Examples 4 and 5 was measured in a transactivation test. The activation of receptors by an agonist (activator) in HeLa cells leads to the expression of a reporter gene, such as luciferase, which generates light in the presence of a substrate. The activation of receptors can accordingly be measured by quantifying the luminescence produced after incubation of the cells in the presence of a reference antagonist. The agonists will displace the antagonist from its binding site producing an increase in luminescence which can be quantified.

Determination of AC₅₀.

To determine the AC50s (concentration at which 50% activation is observed) of 6-[3-(adamantan-1-yl)-4-(prop-2-ynyloxy)phenyl]naphthalene-2-carboxylic acid and 5-[(E)-3-oxo-3-(5,5,8,8-tetrahydronaphthalene-2-yl)propenyl]thiophene-2-carboxylic acid, a 96-well plate assay to generate “crossed curves” was used. The products were tested against a reference antagonist (4-(5,5-dimethyl-8-p-tolyl-5,6-dihydronaphthalen-2-ylethynyl)-benzoic acid). Ten concentrations of the product to be tested plus one zero concentration were placed in rows and seven concentrations of the antagonist plus a zero concentration were placed in columns. This represents 88 measurement points for 1 product and 1 receptor. The remaining 8 wells are used for 100% controls (4-[2-(5,5,8,8-tetramethyl-5,6,7,8-tetrahydronaphthalene-2-yl)propenyl]-benzoic acid) and 0% controls (4-[(E)-3-[4-(4-tert-butyl-phenyl)-5,5,8,8-tetramethyl-5,6,7,8-tetrahydronaphthalen-2-yl]-3-oxo-propenyl)-benzoic acid).

The use of crossed curves enables the determination of the AC₅₀ of the reference ligand at different concentrations of the product to be tested. The AC₅₀s are used to calculate the Schild regression by drawing a straight line corresponding to the Schild equation (“Quantitation in Receptor Pharmacology,” Terry P. Kenakin, Receptors and Channels 7:371-385 (2001)).

The HeLa cell lines used are stable transfectants containing the plasmids ERE-Glob-Luc-SV-Neo (reporter gene) and RAR (α,β,γ)ER-DBD-puro. These cells were seeded in 96-well plates (10,000 cells per well) in 100 μl of DMEM medium without phenol red and supplemented with 10% delipidized calf serum. The plates were incubated at 37° C., 7% CO₂ for four hours.

The products to be tested, the reference antagonist, the 100% control, and the 0% control were added in the amount of 5 μl per well. The plates were then incubated for 18 hours at 37° C., 7% CO₂. The culture medium was eliminated by turning the plate upside down and 100 μl of a 1:1 mixture of PBS/luciferin was added to each well. After 5 minutes, the plates were read by the luminescence reader.

The data generated are shown in the following table.

| | RECEPTOR AC₅₀ (nM) | | |
|---|---|---|---|
| Compound | RARα | RARβ | RARγ |
| 6-[3-(adamantan-1-yl)-4-(prop-2- | 15 | 0.5 | 8 |
| ynyloxy)phenyl]naphthalene-2-carboxylic | | | |
| acid | | | |
| 5-[(E)-3-oxo-3-(5,5,8,8-tetrahydro- | 30 | 60 | 120 |
| naphthalene-2-yl)propenyl]thiophene-2- | | | |
| carboxylic acid | | | |

While the invention has been described in terms of various preferred embodiments, the skilled artisan will appreciate that various modifications, substitutions, omissions and additions may be made without departing from the spirit thereof. Accordingly, it is intended that the scope of the present invention be limited solely by the scope of the following claims.

What is claimed is:

1. A method for increasing the rate of apoptosis in at least one cell type population in which apoptosis is induced by activating receptors of the RAR-γ type, said method comprising exposing said cell population to an effective amount of at least one agonist ligand for receptors of the RAR-γ type to increase the rate of apoptosis, wherein said ligand is 6-[3-(adamantan-1-yl)-4-(prop-2-ynyloxy)phenyl]naphthalene-2-carboxylic acid or 5-[(E)-3-oxo-3-(5,5,8,8-tetrahydronaphthalene-2-yl)propenyl]thiophene-2-carboxylic acid.

2. The method of claim 1, wherein said ligand is 6-[3-(adamantan-1-yl)-4-(prop-2-ynyloxy)phenyl]naphthalene-2-carboxylic acid.

3. The method of claim 1, wherein said agonist ligand is 5-[(E)-3-oxo-3-(5,5,8,8-tetrahydronaphthalene-2-yl)propenyl]thiophene-2-carboxylic acid.

4. The method of claim 1, which is used to treat diseases or disorders linked to an inadequate rate of apoptosis.

5. The method of claim 2, which is used to treat diseases or disorders linked to an inadequate rate of apoptosis.

6. The method of claim 3, which is used to treat diseases or disorders linked to an inadequate rate of apoptosis.

7. The method of claim 4, wherein said agonist ligand is used in combination with a pharmaceutically acceptable carrier.

8. The method of claim 5, wherein said agonist ligand is used in combination with a pharmaceutically acceptable carrier.

9. The method of claim 6, wherein said agonist ligand is used in combination with a pharmaceutically acceptable carrier.

10. The method of claim 4, wherein said agonist ligand is used in combination with a cosmetically acceptable carrier.

11. The method of claim 5, wherein said agonist ligand is used in combination with a cosmetically acceptable carrier.

12. The method of claim 6, wherein said agonist ligand is used in combination with a cosmetically acceptable carrier.

13. A method for the treatment of a skin disorder due to ultraviolet radiation exposure and/or preventing and/or combating photo-induced or chronological aging of the skin and/or reducing actinic keratoses or pigmentations and/or pathology associated with chronological or actinic aging, comprising applying to the skin of an individual in need of such treatment an effective amount of at least one agonist ligand for RAR receptors, wherein said agonist ligand is 6-[3-(adamantan-1-yl)-4-(prop-2-ynyloxy)phenyl]napthalene-2-carboxylic acid or 5-[(E)-3-oxo-3-(5,5,8,8-tetrahydronaphthalene-2-yl)propenyl]thiophene-2-carboxylic acid.

14. The method of claim 13, wherein said agonist ligand is used in combination with a pharmaceutically acceptable carrier.

15. The method of claim 13, wherein said agonist ligand is used in combination with a cosmetically acceptable carrier.

16. A method for the treatment of a dermatological complaint associated with a keratinization disorder characterized by abnormal differentiation and proliferation comprising administering to an individual in need of such treatment at an effective amount of least one agonist ligand for RAR receptors, wherein said agonist ligand is 6-[3-(adamantan-1-yl)-4-(prop-2-ynyloxy)phenyl]napthalene-2-carboxylic acid or 5-[(E)-3-oxo-3-(5,5,8,8-tetrahydronaphthalene-2-yl)propenyl]thiophene-2-carboxylic acid.

17. The method of claim 16, wherein said agonist ligand is used in combination with a pharmaceutically acceptable carrier.

18. The method of claim 16, wherein said agonist ligand is used in combination with a cosmetically acceptable carrier.

19. The method of claim 16, wherein said dermatological complaint associated with a keratinization disorder characterized by abnormal differentiation and proliferation is simple acnes, comedones, polymorphonuclear leukocytes, rosacea, nodulocystic acne, acne conglobata, senile acne, or secondary acne.

20. A method for the treatment of a dermatological complaint with an inflammatory immunoallergenic component, with or without an abnormality of cell proliferation, immune dermatitis, lupus erythematosus, immune bullosis, collagen diseases, or a dermatological or general complaint with an immunological component, comprising administering to an individual in need of such treatment an effective amount of at least one agonist ligand for RAR receptors, wherein said agonist ligand is 6-[3-(adamantan-1-yl)-4-(prop-2-ynyloxy)phenyl]napthalene-2-carboxylic acid or 5-[(E)-3-oxo-3-(5,5,8,8-tetrahydronaphthalene-2-yl)propenyl]thiophene-2-carboxylic acid.

21. The method of claim 20, wherein said agonist ligand is used in combination with a pharmaceutically acceptable carrier.

22. The method of claim 20, wherein said agonist ligand is used in combination with a cosmetically acceptable carrier.

23. The method of claim 20, wherein the dermatological complaint is psoriasis, psoriatic rheumatism, cutaneous atopy, or gingival hypertrophy.

24. The method of claim 23, wherein the psoriasis is cutaneous, mucous, or ungual psoriasis.

25. A method for the treatment of a dermal or epidermal atrophy produced by local or general administration of corticosteroid or other cutaneous atrophy, comprising administering to an individual in need of such treatment an effective amount of at least one agonist ligand for RAR receptors, wherein said agonist ligand is 6-[3-(adamantan-1-yl)-4-(prop-2-ynyloxy)phenyl]napthalene-2-carboxylic acid or 5-[(E)-3-oxo-3-(5,5,8,8-tetrahydronaphthalene-2-yl)propenyl]thiophene-2-carboxylic acid.

26. The method of claim 25, wherein said agonist ligand is used in combination with a pharmaceutically acceptable carrier.

27. The method of claim 25, wherein said agonist ligand is used in combination with a cosmetically acceptable carrier.

28. A method for the treatment of a pigmentary disorder, comprising administering to an individual in need of such treatment an effective amount of at least one agonist ligand for RAR receptors, wherein said agonist ligand is 6-[3-(adamantan-1-yl)-4-(prop-2-ynyloxy)phenyl]napthalene-2-carboxylic acid or 5-[(E)-3-oxo-3-(5,5,8,8-tetrahydronaphthalene-2-yl)propenyl]thiophene-2-carboxylic acid.

29. The method of claim 28, wherein said agonist ligand is used in combination with a pharmaceutically acceptable carrier.

30. The method of claim 28, wherein said agonist ligand is used in combination with a cosmetically acceptable carrier.

31. The method of claim 28, wherein said pigmentary disorder is hyperpigmentation, hypopigmentation, or vitiligo.

32. A method for the treatment of prevention of alopecia, comprising administering to an individual in need of such treatment or prevention an effective amount of at least one agonist ligand for RAR receptors, wherein said agonist ligand is 6-[3-(adamantan-1-yl)-4-(prop-2-ynyloxy)phenyl]napthalene-2-carboxylic acid or 5-[(E)-3-oxo-3-(5,5,8,8-tetrahydronaphthalene-2-yl)propenyl]thiophene-2-carboxylic acid.

33. The method of claim 32, wherein said agonist ligand is used in combination with a pharmaceutically acceptable carrier.

34. The method of claim 32, wherein said agonist ligand is used in combination with a cosmetically acceptable carrier.

35. The method of claim 32, wherein the alopecia is due to chemotherapy or radiation.

36. The compound having the formula:

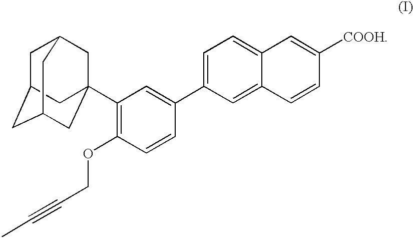

37. A composition comprising the compound of claim 36, in an amount effective to increase the rate of apoptosis in at least one cell type population in which apoptosis is induced by activating receptors of the RAR-γ type and a pharmaceutically acceptable carrier.

38. A composition comprising the compound of claim 36, in an amount effective to increase the rate of apoptosis in at least one cell type population in which apoptosis is induced by activating receptors of the RAR-γ type and a cosmetically acceptable carrier.

39. A composition comprising the compound of claim 36, in an amount effective to activate receptors of the RAR-α or RAR-β and a pharmaceutically acceptable carrier.

40. A composition comprising the compound of claim 36, in an amount effective to activate receptors of the RAR-α or RAR-β and a cosmetically acceptable carrier.

41. The compound having the formula:

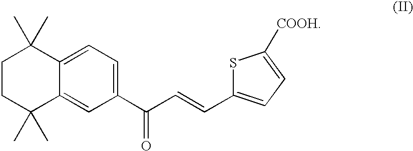

42. A composition comprising the compound of claim 41, in an amount effective to increase the rate of apoptosis in at least one cell type population in which apoptosis is induced by activating receptors of the RAR-γ type and a pharmaceutically acceptable carrier.

43. A composition comprising the compound of claim 41, in an amount effective to increase the rate of apoptosis in at least one cell type population in which apoptosis is induced by activating receptors of the RAR-γ type and a cosmetically acceptable carrier.

44. A composition comprising the compound of claim 41, in an amount effective to activate receptors of the RAR-α or RAR-β type and a pharmaceutically acceptable carrier.

45. A composition comprising the compound of claim 41, in an amount effective to activate receptors of the RAR-α or RAR-β and a cosmetically acceptable carrier.
